(19) United States
(12) Patent Application Publication
(10) Pub. No.: US 2003/0108461 A1
(43) Pub. Date: Jun. 12, 2003

(54) Aroma lamp

(76) Inventor: Hsien-Jen Lo, Tu-Cheng City (TW)

Correspondence Address: RABIN & BERDO, P.C., 1101 14th Street, N.W., Suite 500, Washington, DC, 20005, US

(21) Appl. No.: 10/004,408

(22) Filed: Dec. 6, 2001

(51) Int. Cl. A61L 9/00

(52) U.S. Cl. 422/305; 422/306; 422/307

(57) ABSTRACT

An inner-burn aroma lamp mainly includes a perfume essence cup overlapped on the upper part of a lamp pot, whereas at the center thereof the perfume essence cup having a center tube while on the top end of the center tube perforated through by a lamp wick frame which is traversed by a lamp wick extended perpendicularly to the bottom of the lamp pot. Accordingly, the lamp oil is filled into the lamp pot, and perfume essence is filled into perfume essence cup so that the lamp wick could siphon the burning oil and heat the perfume essence cup. Therefore the air near the perfume cup is warmed so as to vaporize the perfume essence in the perfume essence cup.

### BACKGROUND OF THE INVENTION

1. Field of the Invention

The present invention relates to an inner-burn aroma lamp, more particular, to a kind of aroma lamp that vaporizes perfume essence by the heat energy from hot air and the heat conducted from the cup.

2. Description of the Prior Art

Accordingly, in order to improve air quality in some situation, it is often to heat the perfume essence, emitting fragrant order to clear the air and to create a mood of quiet and pleasant, and reach some extent of emotional comfort, relax, and spiritual pleasure.

Whereas, the conventional method of vaporizing perfume essence is to fill perfume essence on the top of a metal or porcelain cup, and under the metal or porcelain cup fixed a candle or alcohol lamp, by burning the candle or alcohol to heat the metal or porcelain cup, the perfume essence in the cup is heated and diffused over a length of time.

By burning the candle or alcohol lamp underneath as hot source, the perfume essence filled in the heat-resistant container is heated directly; such method is very similar to the way of cook. At the very beginning of inflaming the candle or alcohol lamp, the heat-resistant container of perfume essence is heated at first, the perfume essence does not get hot at this moment. After a period of burning time passed, the heat-resistent container takes a certain volume of heat, then the perfume essence start to gather heat and vaporize. Once too much of heat is taken by heat-resistant container, the perfume essence is vaporized like boiling. The more heat the perfume essence absorbs, the faster the perfume essence vaporizes. Because the fast heating of perfume essence makes perfume essence vaporize in a short time and concurrently produce strong perfume order full in the air. The perfume essence is burned at a different heat intensity depending on different containers.

Additionally, in order to avoid strong perfume chocks in the air, the candle or alcohol lamp must be removed periodically from the container for perfume essence, and then fire again and put the candle or alcohol lamp back. The back and forth of the candle or alcohol lamp from the container therefore cause inconvenience and bother the users. Moreover, there are another kind of lamp using electro-thermal wire to heat the perfume essence container, and make the perfume essence hot and vaporize; but the electro-thermal wire spend large volume of electricity and delicate enough to be damaged. Its no water-resistant structure tends to get short when penetrate by water. Thus it's not practical.

### SUMMARY OF THE INVENTION

It is therefore a primary object of the present invention to provide an aroma lamp that vaporizes perfume essence by the heat energy from hot air and the heat conducted by the container in order to diffuse fragrant order.

In order to improve the above-mentioned defectives of conventional perfume essence lamp and achieve the above object, an aroma lamp includes a perfume essence cup overlapped on the upper part of a lamp pot. While at the center of the perfume essence cup having a center tube, on the top end of which is perforated through by a lamp wick frame that is traversed by a lamp wick extended perpendicularly to the bottom of the lamp pot. According to the structure mentioned above, the lamp oil is filled into the lam pot, and perfume essence sits on the perfume essence cup so that the lamp wick could siphon the lamp oil and inflame at the perfume essence cup. Therefore, the perfume essence cup itself and the air nearby is heated and the perfume essence is simmered to release fragrant smell.

### BRIEF DESCRIPTION OF THE DRAWINGS

The accomplishment of the above-mentioned object of the present invention will become apparent from the following description and its accompanying drawings which disclose illustrative an embodiment of the present invention, and are as follows.

FIG. 1 is a perspective view of a mini earphone socket in accordance with the present invention;

FIG. 2 is an explosive view of the present invention;

FIG. 3 is a schematic drawing of the sectional view of the present invention;

FIG. 4 is a perspective view of another embodiment in accordance with the present invention;

FIG. 5 is an explosive view of the FIG. 4;

FIG. 6 is a sectional view of the FIG. 4;

FIG. 7 is a schematic drawing of the sectional view of another embodiment in accordance with the present invention.

### DETAILED DESCRIPTION OF THE PREFERRED EMBODIMENT

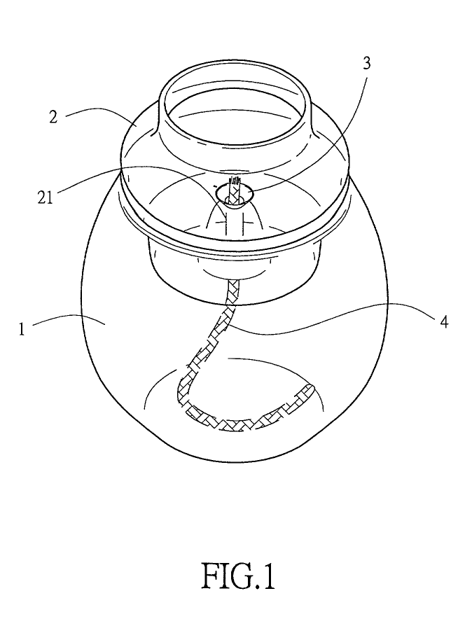

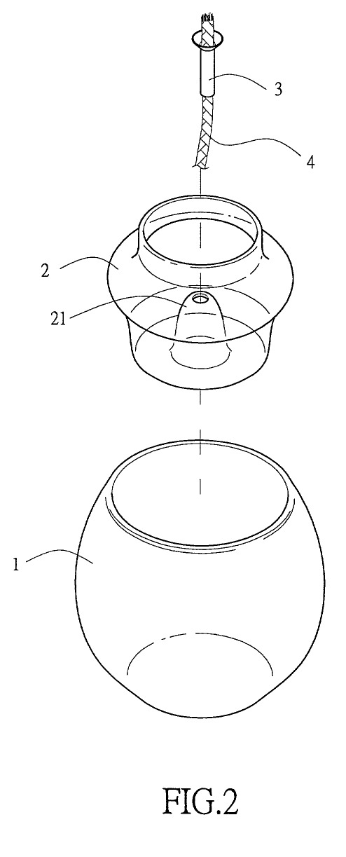

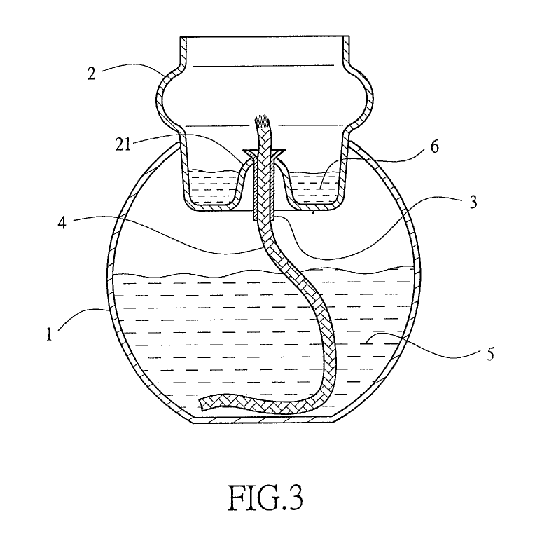

First, refer to FIGS. 1 and 2, the invention which is composed of a perfume essence cup 2 overlapped on the upper part of a lamp pot 1, whereas a center tube 21 is arranged at the center of the perfume essence cup 2. On the top end of the center tube 21, a lamp wick frame 3 is perforated through, which is traversed by a lamp wick 4 extended perpendicularly to the bottom of the lamp pot 1. Accordingly, lamp oil 5 is filled into the lamp pot 1, and perfume essence 6 is filled into perfume essence cup 2, as shown on FIG. 3, so that the lamp wick 4 could absorb the lamp oil 5 and is burned for heating the perfume essence cup 2. Therefore the warmed air nearby, and the heat conducted from heat source, through the lamp wick frame 3 fitted on the top of the center tube 21 to the perfume essence cup 2, both transmit heat energy and vaporize the perfume essence 6 in the perfume essence cup 2.

Because the lamp wick 4 is inserted through the center tube 21 perforated through the lamp wick frame 3, only at the top of perfume essence cup 2 that part of the lamp wick 4 is burned. The flame does not heat the perfume essence 6 directly, but fill the hot air in the perfume essence cup 2. The perfume essence 6 is simmered by hot air near the cup 2 and the heat conducted through the perfume essence cup 2. Thus not only the heat transmit speed is lowered but also adequate heat is offered for vaporization. Therefore, the period of volatilizing perfume essence 6 is extended and the problem of strong order is avoided. This is a better way to fragrance the environment.

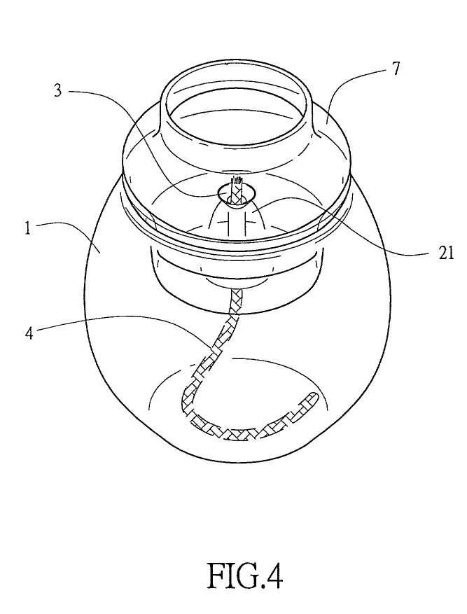

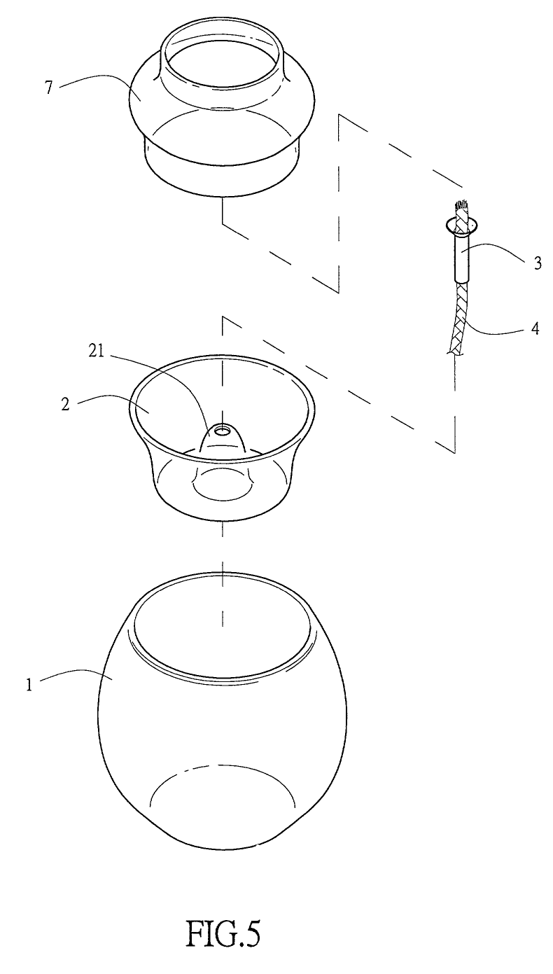

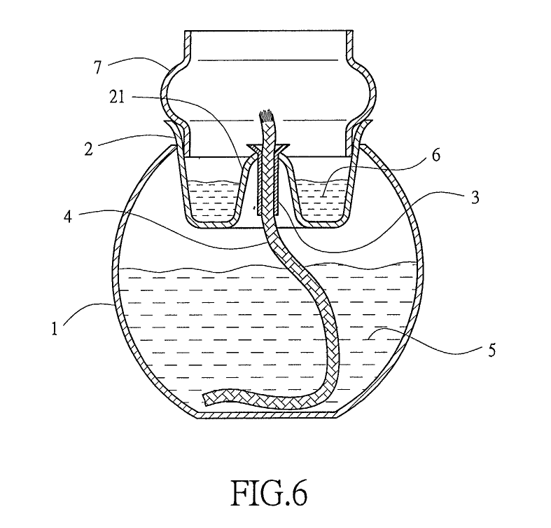

Please also refer to FIG. 4, FIG. 5 and FIG. 6, the perfume essence cup 2 is an open-mouthed cup and could be covered by a lampshade 7 and also achieve above-mentioned effect. This is another embodiment of the present invention.

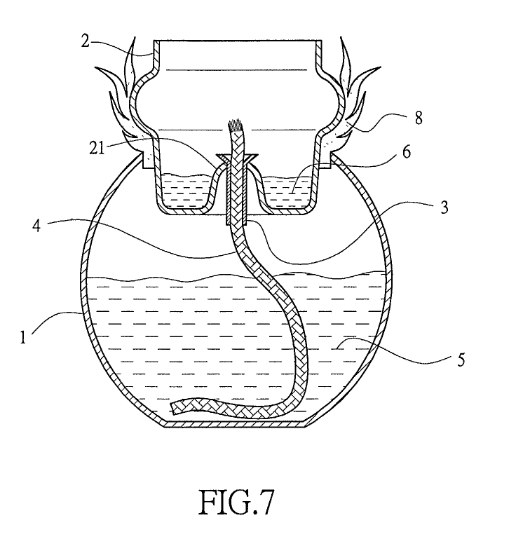

Please also refer to FIG. 7, a decorative ring 8 is disposed between the lamp mouth of the lamp pot 1 and the perfume essence cup 2 for changing the outlook of the lamp in various way and adding a decorative touch. This is a further embodiment of the present invention.

Additionally, the length of lamp wick 4 above the lamp frame 3 could be adjusted in order to reset the size of the flame. The bigger the flame is, the more heat the perfume essence 6 gains, then the perfume essence vaporize soon; and vice versa.

Many changes and modifications in the above-described embodiments of the invention can, of course, be carried out without departing from the scope thereof. Accordingly, to promote the progress in science and the useful arts, the invention is disclosed and is intended to be limited only by the scope of the appended claim.

What is claimed is:

1. An Aroma lamp comprising a perfume essence cup, with a center tube arranged at the center thereof, arranged on the top of a lamp pot, a lamp wick frame perforated through said center tube, a lamp wick traversed said lamp wick frame and extended perpendicularly to the bottom of said lamp pot;
wherein after perfume essence is filled into said perfume essence cup and lamp oil is filled into said lamp pot, said lamp wick absorbs the lamp oil and is burned for heating said perfume essence cup and air nearby in order to simmer the perfume essence.

2. An aroma lamp as claimed in claim 1, wherein said perfume essence cup can be an open-mouthed cup and is covered by a lampshade.

3. An aroma lamp as claimed in claim 1, wherein a decorative ring is disposed between the lamp mouth of said lamp pot and said perfume essence cup.
